SEQUENCE LISTING (4 sequences)

SEQ ID NO: 1 - protein, 39 aa, synthetic construct
  SITE 2: note = X at position 2 is Aib
  SITE 13: note = X at position 13 is Aib
  MOD_RES 20: note = Lys at position 20 is chemically modified through conjugation to the epsilon-amino group of the K side-chain with (2-[2-(2-Amino-ethoxy)-ethoxy]-acetyl)2-(gamma-Glu)1- CO-(CH2)18-CO2H
  MOD_RES 39: note = C-terminal amino acid is amidated as a C-terminal primary amide
YXEGTFTSDYSIXLDKIAQKAFVQWLIAGGPSSGAPPPS

SEQ ID NO: 2 - protein, 39 aa, synthetic construct; identical to SEQ ID NO: 1
  SITE 2: note = X at position 2 is Aib
  SITE 13: note = X at position 13 is Aib
  MOD_RES 39: note = C-terminal amino acid is optionally amidated as a C-terminal primary amide
  MOD_RES 20: note = K at position 20 is chemically modified through conjugation to the epsilon-amino group of the K side-chain with a C16-C20 fatty acid or a derivative thereof

SEQ ID NO: 3 - protein, 39 aa, synthetic construct; identical to SEQ ID NO: 1
  SITE 2: note = X at position 2 is Aib
  SITE 13: note = X at position 13 is Aib
  MOD_RES 39: note = C-terminal amino acid is optionally amidated as a C-terminal primary amide
  MOD_RES 20: note = K at position 20 is chemically modified through conjugation to the epsilon-amino group of the K side-chain with a fatty acid selected from the group consisting of (2-[2-(2-Amino-ethoxy)-ethoxy]-acetyl)2-(gamma-Glu)1-CO-(C H2)18-PO(OH)2, (2-[2-(2-Amino-ethoxy)-ethoxy]-acetyl)2-CH2 CH2-CHPO(OH)2-CO-(CH2)18-CO2H, and (2-[2-(2-Amino-ethoxy)- ethoxy]-acetyl)2-CO-CH2CH2-CHPO(OH)2-CO-(CH2)18-PO(OH)2

SEQ ID NO: 4 - protein, 39 aa, synthetic construct; identical to SEQ ID NO: 1
  SITE 2: note = X at position 2 is Aib
  SITE 13: note = X at position 13 is Aib
  MOD_RES 39: note = C-terminal amino acid is amidated as a C-terminal primary amide
  MOD_RES 20: note = K at position 20 is chemically modified through conjugation to the epsilon-amino group of the K side-chain with (2-[2-(2-Amino-ethoxy)-ethoxy]-acetyl)-(Aib)-(gamma-G lu)-CO-(CH2)18-CO2H

(19) United States
(12) Patent Application Publication
(10) Pub. No.: US 2026/0130970 A1
(43) Pub. Date: May 14, 2026

(54) PRESERVED GIP/GLP AGONIST COMPOSITIONS

(71) Applicant: ELI LILLY AND COMPANY, Indianapolis, IN (US)

(72) Inventors: David Paul ALLEN, Carmel, IN (US); Brandon Lee DOYLE, Indianapolis, IN (US); Evan Michael HETRICK, Carmel, IN (US); Ken Kangyi QIAN, Carmel, IN (US)

(21) Appl. No.: 19/122,313

(22) PCT Filed: Oct. 18, 2023
(86) PCT No.: PCT/US2023/077124

§ 371 Date: Apr. 18, 2025

Related U.S. Application Data
(60) Provisional application No. 63/417,339, filed on Oct. 19, 2022.
(60) Provisional application No. 63/380,998, filed on Oct. 26, 2022.

(51) Int. Cl. A61K 38/22; A61K 47/02; A61K 47/10

(52) U.S. Cl. CPC A61K 38/2235; A61K 47/02; A61K 47/10

(57) ABSTRACT

A preserved composition of tirzepatide, comprising less than or equal to about 3 mg/mL NaCl; phosphate buffer, phenol, benzyl alcohol and glycerin.

The present invention is a preserved pharmaceutical GIP/GLP1 dual-agonist peptide composition for subcutaneous injection. The composition comprises an antimicrobial preservative and excipients to control undesired oligomerization and provide desired stability. The composition comprises tirzepatide, NaCl, glycerin, phenol, benzyl alcohol, and phosphate buffer. The composition provides commercially acceptable shelf-life stability, in-use stability, and pharmaceutically desirable control of peptide oligomerization.

Diabetes mellitus is a chronic disorder characterized by hyperglycemia resulting from defects in insulin secretion, insulin action, or both. In type 2 diabetes mellitus (“T2D”), the combined effects of impaired insulin secretion and insulin resistance are associated with elevated blood glucose levels. Tirzepatide is a GIP/GLP1 dual-agonist peptide useful in the treatment of diabetes. After its FDA approval in May 2022, tirzepatide is sold in the United States under the brand name Mounjaro™. Tirzepatide is useful in the treatment of obesity. Pharmaceutically acceptable multiuse compositions are desired to enable alternate device and delivery options. A composition suitable for multiuse delivery generally requires an antimicrobial agent to preserve the composition during multiple uses. Benzyl alcohol and phenol are preservatives that may be used in a multiuse formulation; however, preservatives are reported to interact with biologics. Interaction between biologics and preservatives may result in oligomerization, cloudiness, instability, and other undesired effects. There is a need for a stable, pharmaceutically acceptable, preserved incretin formulation.

U.S. Pat. No. 9,474,780 generally describes compositions containing a GIP/GLP1 agonist, administered by parenteral routes. U.S. Pat. No. 9,474,780 describes and claims tirzepatide. U.S. Pat. No. 11,357,820 describes and claims compositions for pharmaceutically desirable single-use presentation, for example, in a single-use pen. U.S. Pat. No. 11,357,820 discloses a preserved formulation; however, applicants discovered tirzepatide is subject to self-association or oligomerization when preserved in the presence of 4 mg/mL or greater NaCl using typical pharmaceutical preservatives. Self-association or oligomerization of tirzepatide may impact stability, shelf life, and properties of the peptide. There is a desire for preserved compositions of tirzepatide providing acceptable stability, shelf life, and pharmaceutically desirable control of peptide oligomerization.

### BRIEF DESCRIPTION OF THE DRAWINGS

FIG. 1. Static light scattering of tirzepatide in solution containing 140 mM NaCl, pH 7.0.

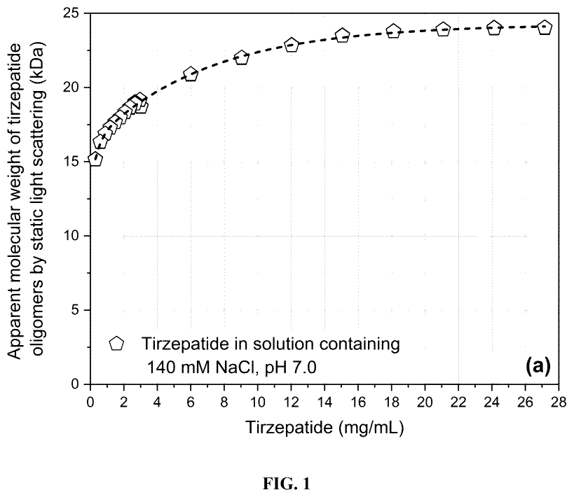

FIG. 2. Static light scattering of tirzepatide in solution containing 140 mM NaCl and 5 mg/mL phenol, pH 7.0 and tirzepatide in solution containing 140 mM NaCl, pH 7.0.

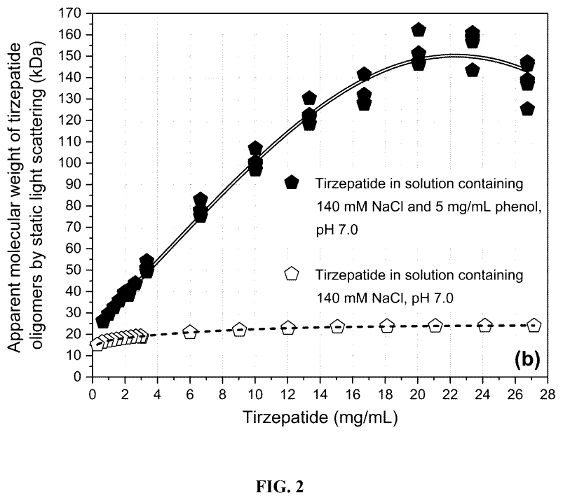

FIG. 3. Static light scattering of tirzepatide in 200 mM NaCl; 140 mM NaCl; 85 mM NaCl; 30 mM NaCl; phenol with 0 mM NaCl; glycerin with 0 mM NaCl; and 0 mM NaCl.

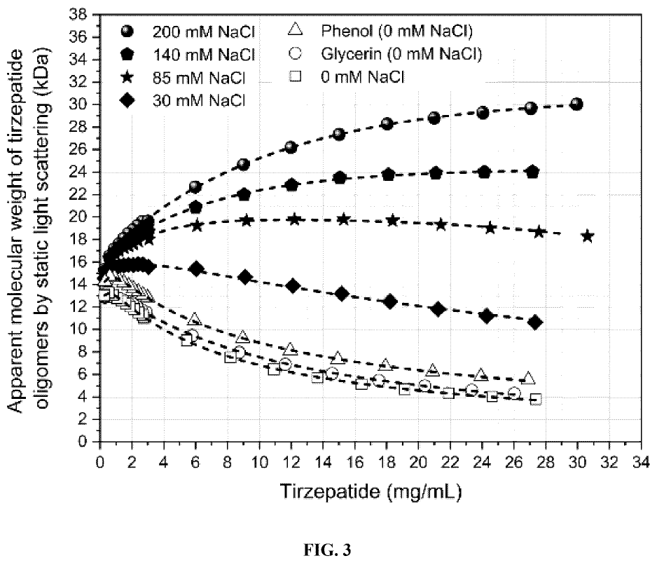

FIG. 4. Static light scattering of tirzepatide in 30 mM NaCl and 8 mg/mL phenol; 30 mM NaCl and 5 mg/mL phenol; 30 mM NaCl; 8 mg/mL phenol with 0 mM NaCl; and 5 mg/mL phenol with 0 mM NaCl.

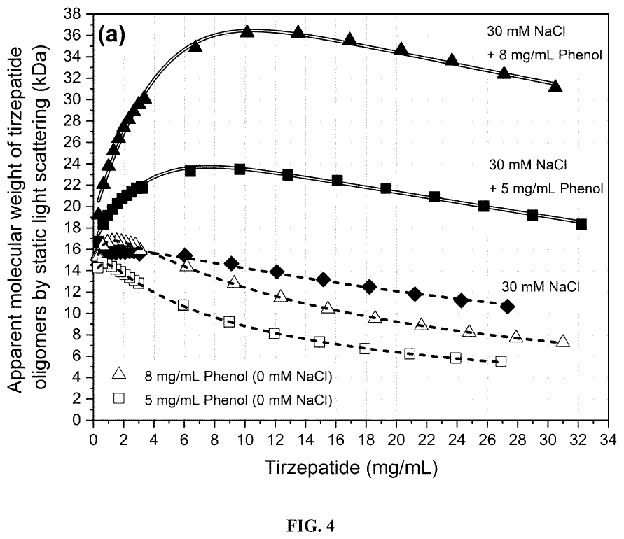

FIG. 5. Static light scattering of tirzepatide in 30 mM NaCl with 15 mg/mL benzyl alcohol; 30 mM NaCl with 9 mg/mL benzyl alcohol; 30 mM NaCl; 15 mg/mL benzyl alcohol with 0 mM NaCl; and 9 mg/mL benzyl alcohol with 0 mM NaCl.

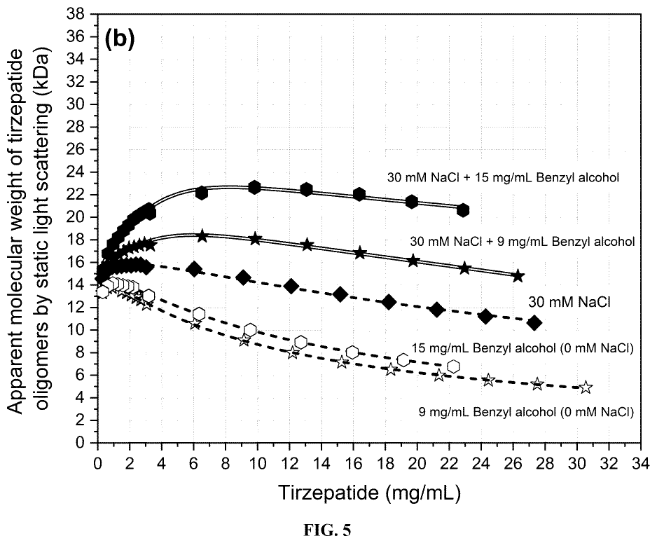

FIG. 6. Static light scattering of tirzepatide with 30 mM NaCl, 9 mg/mL benzyl alcohol, and 2 mg/mL phenol; 30 mM NaCl; and 9 mg/mL benzyl alcohol with 2 mg/mL phenol and 0) mM NaCl.

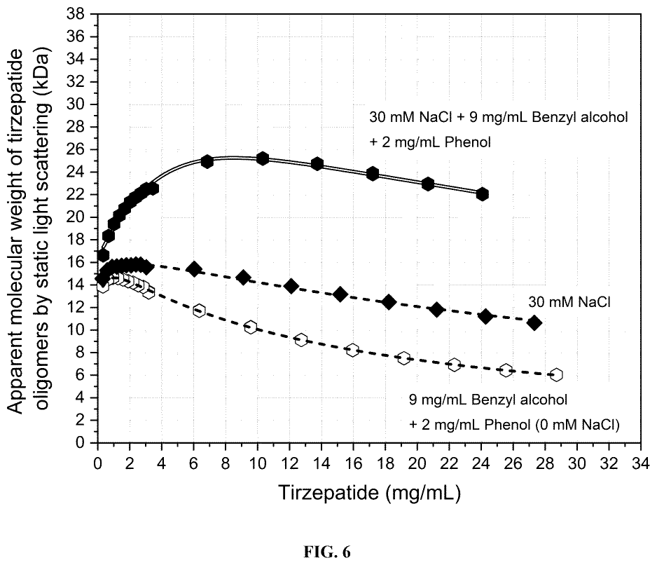

FIG. 7. Ligand-observed NMR of preservatives. In upper left is 1.8 mg/mL benzyl alcohol; upper right is 1.8 mg/mL benzyl alcohol plus 1 mg/mL tirzepatide; Lower left: 1.5 mg/mL phenol; Lower right: 1.5 mg/mL phenol plus 1.0 mg/mL tirzepatide.

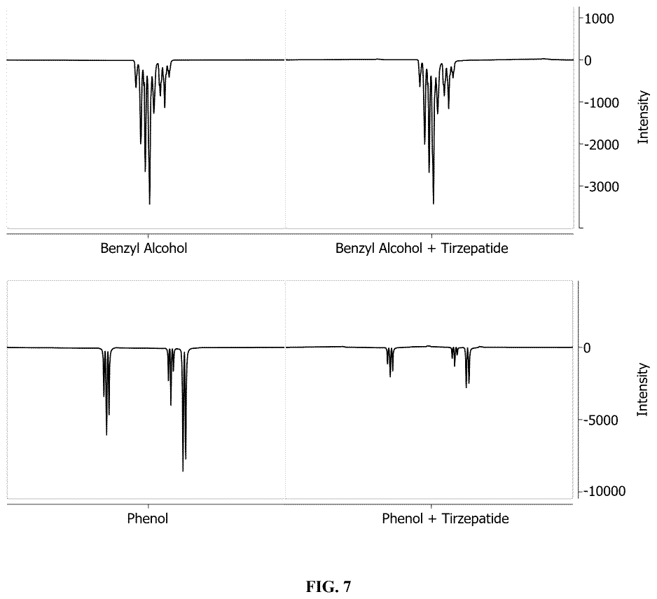

FIG. 8. ¹H-¹³C HSQC spectrum of tirzepatide (black) overlayed with that of tirzepatide with benzyl alcohol. No significant differences between the spectra are observed.

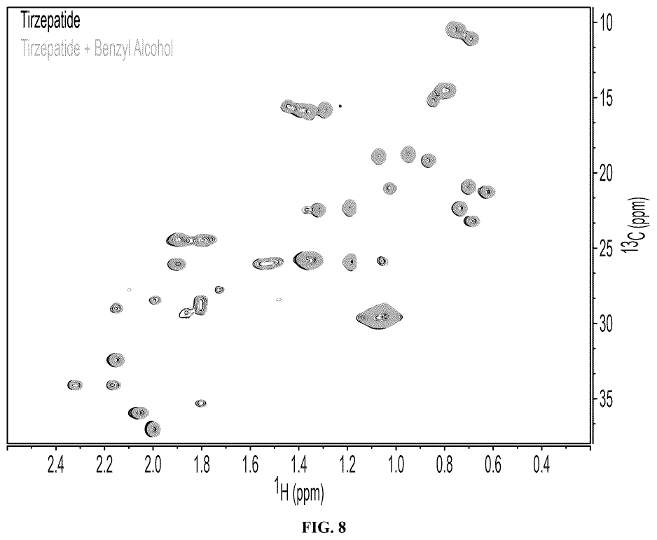

FIG. 9. ¹H-¹³C HSQC spectrum of tirzepatide (black) overlayed with that of tirzepatide with phenol. Significant differences between the spectra are observed showing interaction of phenol with tirzepatide.

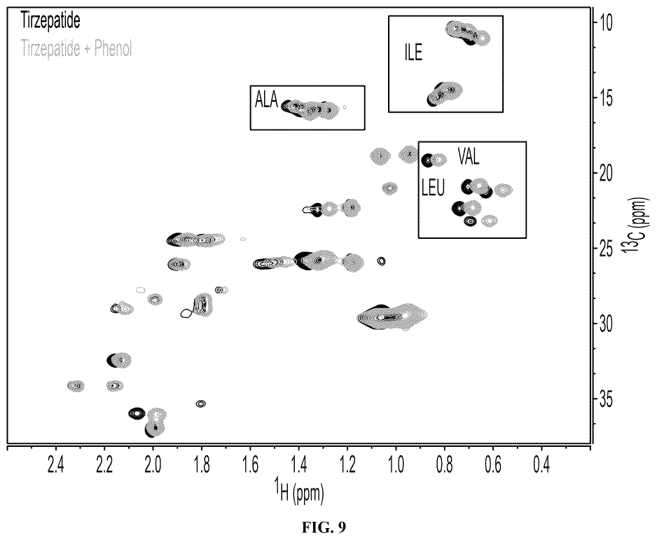

Applicants discovered that NaCl concentration is an important aspect involved in tirzepatide self-association.

In an embodiment is a preserved formulation wherein the oligomerization state of tirzepatide is considered equivalent, in accordance with United States regulatory standards, to the Mounjaro™ matrix. In an embodiment is a preserved tirzepatide formulation having a low risk of fibril formation.

Compositions herein seek to meet these needs by providing pharmaceutically-acceptable compositions comprising tirzepatide, or a pharmaceutically acceptable salt thereof; NaCl; glycerin; phenol; benzyl alcohol; and phosphate buffer; wherein the NaCl concentration is less than or equal to about 3 mg/mL.

In an embodiment, NaCl concentration is from about 1.5 mg/mL to about 3 mg/mL. In an embodiment, NaCl is about 25 mM to about 50 mM. In an embodiment NaCl concentration is about 30 mM. In an embodiment, NaCl concentration is about 1.75 mg/mL. In an embodiment, NaCl is a tonicity agent. In an embodiment, NaCl and glycerin serve as a tonicity agent. In an embodiment, is composition wherein glycerin concentration is from about 8 mg/mL to about 12 mg/mL. In an embodiment, NaCl concentration is about 1.75 mg/mL and glycerin concentration is about 8 mg/mL. In an embodiment, NaCl concentration is about 50 mM and glycerin is about 8 mg/mL. In an embodiment NaCl concentration is about 30 mM and glycerin concentration is about 8 mg/mL. In an embodiment NaCl concentration is about 50 mM and glycerin concentration is about 12 mg/mL. In an embodiment, phenol concentration is from about 2 mg/mL to about 5 mg/mL. In an embodiment, phenol concentration is greater than about 5.5 mg/mL. In an embodiment phenol concentration is greater than about 5.5 mg/mL. In an embodiment, the preservative consists of phenol and benzyl alcohol. In an embodiment, the preservative comprises phenol and benzyl alcohol. In an embodiment, the preservative comprises phenol and about 9 mg/mL benzyl alcohol. In an embodiment, the preservative is about 2 mg/mL phenol and about 9 mg/mL benzyl alcohol. In an embodiment, phenol concentration is less than about 6 mg/mL.

In an embodiment, the phosphate buffer is dibasic sodium phosphate. In an embodiment, the phosphate buffer concentration is from about 0.67 mg/mL to about 2.0 mg/mL. In an embodiment, the phosphate buffer concentration is about 1.34 mg/mL. In an embodiment, phosphate buffer is about 5 mM.

In an embodiment, the tirzepatide concentration is from about 2.09 mg/mL to about 41.67 mg/mL. In an embodiment, the tirzepatide, or a pharmaceutically acceptable salt thereof, concentration is from about 4.17 mg/mL to about 25.0 mg/mL. In an embodiment, the tirzepatide, or pharmaceutically acceptable salt thereof, is from about 4.17 mg/mL to about 33.4 mg/mL. In an embodiment, the tirzepatide, or pharmaceutically acceptable salt thereof, is from about 4.17 mg/mL to about 41.67 mg/mL. In an embodiment, the tirzepatide, or pharmaceutically acceptable salt thereof is from about 10 mg/mL to about 30 mg/mL. In an embodiment the tirzepatide, or a pharmaceutically acceptable salt thereof, concentration is from about 5 mg/mL to about 50.0 mg/mL. In an embodiment, the tirzepatide, or a pharmaceutically acceptable salt thereof, concentration is from about 2.5 mg/mL to about 30 mg/mL. In an embodiment about 0.6 mL is delivered per dose administration. In an embodiment, about 0.5 mL is delivered per dose administration.

The tirzepatide, or pharmaceutically acceptable salt thereof, concentration may be adjusted to deliver the desired dose per injection. For example, when about 0.6 mL volume per dose is administered, the concentration of tirzepatide, or a pharmaceutically acceptable salt thereof, will be adjusted to ensure accurate delivery of the desired tirzepatide, or a pharmaceutically acceptable salt thereof, dose. In an embodiment, the tirzepatide, or a pharmaceutically acceptable salt thereof, dose is selected from the group consisting of 2.5 mg, 5 mg, 7.5 mg, 10 mg, 12.5 mg, and 15 mg. In an embodiment the tirzepatide, or a pharmaceutically acceptable salt thereof, dose is selected from the group consisting of 2.5 mg, 5 mg, 7.5 mg, 10 mg, 12.5 mg, 15 mg, 20 mg, and 25 mg. In an embodiment, the tirzepatide, or a pharmaceutically acceptable salt thereof, dose is selected from the group consisting of 1.25 mg, 2.5 mg, 5 mg, 7.5 mg, 10 mg, 12.5 mg, 15 mg, 20 mg, and 25 mg. In an embodiment, the tirzepatide, or a pharmaceutically acceptable salt thereof, dose is selected from the group consisting of 10 mg and 15 mg. In an embodiment, the dose is selected from the group consisting of 10 mg, 12.5 mg, 15 mg, 20 mg, and 25 mg. In an embodiment, the dose is selected from about 1.25 mg, 2.5 mg and 5 mg.

In an embodiment, the tirzepatide, or a pharmaceutically acceptable salt thereof, concentration is from about 4.17 to about 25 mg/mL and NaCl concentration is about 1.75 mg/mL. In an embodiment, the tirzepatide, or a pharmaceutically acceptable salt thereof, concentration is from about 2.09 mg/mL to about 25 mg/mL and NaCl concentration is about 1.75 mg/mL.

In an embodiment, the dose of a tirzepatide, or a pharmaceutically acceptable salt thereof, composition is administered about once weekly. In an embodiment, the dose of a tirzepatide, or a pharmaceutically acceptable salt thereof, composition is administered once every seven days.

In an embodiment, there is provided a method of treating diabetes comprising administering to a human in need thereof an effective dose of one of the above-described compositions.

In an embodiment, there is provided a method of treating obesity comprising administering to a human in need thereof an effective dose of one of the above-described compositions. In an embodiment, there is provided a method of providing therapeutic weight loss comprising administering to a human in need thereof an effective dose of one of the above-described compositions. In an embodiment, there is provided a method for improving chronic weight management comprising administering to a human in need thereof an effective dose of one of the above-described compositions. In an embodiment, there is provided a method of treating a condition mediated by GIP/GLP1 co-agonist activity comprising administering to a human in need thereof an effective dose of one of the above-described compositions.

In an embodiment, there is provided one of the above-described compositions for use as a medicament.

In an embodiment, there is provided one of the above-described compositions for use in the treatment of diabetes. In an embodiment, there is provided one of the above-described compositions for use in the treatment of obesity.

In an embodiment, there is provided one of the above-described compositions for use in providing therapeutic weight loss. In an embodiment, there is provided one of the above-described compositions for use in providing non-therapeutic weight loss. In an embodiment, there is provided one of the above-described compositions for use in improving chronic weight management.

According to another aspect of the present invention, there is provided an article of manufacture comprising one of the above-described compositions. In certain embodiments, the article of manufacture is a multi-use vial. In certain embodiments, the article of manufacture is a multi-use cartridge. In certain embodiments, the article of manufacture is a multiuse pen. In certain embodiments, the article of manufacture is a pre-filled syringe.

The Mounjaro™ drug product is formulated as a once-weekly, un-preserved single-use subcutaneous injection. Tirzepatide formulated as Mounjaro™ is approved for use in treating type 2 diabetes, with other indications such as obesity are being explored. There is desire to develop a multi-use, preserved tirzepatide, or a pharmaceutically acceptable salt thereof, formulation, such that the treatment can reach and help more patients. A preserved formulation is desired for use in prefilled cartridges, pens, or vials intended for multiuse.

Antimicrobial preservative is added to a formulation to inhibit or kill microorganisms that may be inadvertently introduced into the product. Such compounds typically contain an aromatic ring in their chemical structures, which permits interactions with other molecules. For instance, preservatives with aromatic ring structure have been reported to induce instability in protein formulations, through mechanisms including unfolding of larger proteins with tertiary structure, aggregation of polysorbate in the drug product compositions and the like.

In the single-use drug product Mounjaro™, the composition is 5 mg/mL to 30 mg/mL of tirzepatide in 5 mM phosphate buffer, 140 mM NaCl, pH=7.0. Under this condition, the peptide reversibly self-associates, exhibiting a monomer-trimer-hexamer equilibrium. The equilibrium provided by the single use drug product Mounjaro™ composition may be relevant to product stability and peptide properties. There is a desire to maintain substantially similar monomer-trimer-hexamer peptide self-association equilibrium to ensure consistent patient experience between the unpreserved and preserved formulations.

As illustrated by the Drawings, phenol, benzyl alcohol, and NaCl interact with tirzepatide to varying degrees. Although benzyl alcohol is reported to interact with peptides, Drawings 7, 8, and 9 illustrate no significant interaction with tirzepatide. Further, it is necessary to include adequate preservatives and agents to provide stability and safety during single patient, multi-use conditions. Drawings 1, 2, 3, 4, 5, and 6 illustrate the unpredictable oligomerization of tirzepatide with various excipients and conditions.

As used herein, “tirzepatide” means a GIP/GLP1 dual-agonist peptide as described in U.S. Pat. No. 9,474,780 and described by CAS Registry Number: 2023788-19-2, and as the active pharmaceutical ingredient in the U.S. FDA approved product, Mounjaro™, Tirzepatide is described in Example 1 of U.S. Pat. No. 9,474,780, with the following sequence:

YX₁EGTFTSDYSIX₂LDKIAQKAFVQWLIAGGPSSGAPPPS

wherein X₁ is Aib; X₂ is Aib; K at position 20 is chemically modified through conjugation to the epsilon-amino group of the K side-chain with (2-[2-(2-Amino-ethoxy)-ethoxy]-acetyl)²-(γGlu)₁-CO—(CH₂)₁₈—CO₂H; and the C-terminal amino acid is amidated as a C-terminal primary amide (SEQ ID NO: 1).

When used herein, “pharmaceutically acceptable salt” is well known to the skilled artisan. In an embodiment is a pharmaceutically acceptable salt that is a tirzepatide trifluoroacetate salt. The compositions are sterile when first produced.

The pH of tirzepatide, or a pharmaceutically acceptable salt thereof, compositions herein are typically about 6.5 to 7.5, and may be adjusted using physiologically appropriate acids and bases, as may be required to achieve the desired pH. In an embodiment the pH target is between 6.7 and 7.3. In an embodiment, the pH target is about 7.

In an embodiment, the pH is adjusted using a base to facilitate dissolution in the buffer solution. The addition of an acid to the composition may be required to adjust the pH to the desired pH range. In an embodiment, NaOH is used to facilitate dissolution of tirzepatide, or a pharmaceutically acceptable salt thereof, in a buffer. In an embodiment, HCl is added to adjust the pH of the composition containing the dissolved tirzepatide to the desired pH range.

The compositions of the present invention are typically administered subcutaneously. The compositions are typically administered using a pre-filled, disposable pen, reusable pen with cartridge, or automatic pen injector. The composition may be administered using a multi-use vial or a pump device.

As used herein, “shelf life stability” is measured under controlled conditions at about 5 degrees Celsius. As used herein, the term “in-use stability” refers to the stability of the composition measured under controlled conditions at or about 25 degrees Celsius or at or about 40 degrees Celsius.

In an embodiment, the term “phenol” means phenol liquified, distilled wherein the phenol is about 90% phenol with about 10% water.

As used herein, the term “about” is a range of variability allowed in accordance with applicable regulatory guidelines. In an embodiment, “about” means plus or minus 10% of the stated value. In an embodiment “about” means plus or minus 5% of the stated value. In an embodiment, “about” means plus or minus 2% of the stated value.

As used herein, benzyl alcohol with low peroxide means benzyl alcohol with a peroxide value about ≤5 and/or about ≤1 Japanese Pharmacopoeia (JP) value and/or about <4 parts per million peroxide. In an embodiment, benzyl alcohol with low peroxide rating is used within one week of opening the benzyl alcohol container for the first time. In an embodiment, benzyl alcohol with low peroxide rating is used within one day of opening the benzyl alcohol container for the first time. In an embodiment, benzyl alcohol with low peroxide rating is stored under refrigeration, and used within 6 months after first opening of the container. In an embodiment, benzyl alcohol with low peroxide rating is stored with nitrogen overlay and used within 6 months from first opening. In an embodiment, benzyl alcohol with low peroxide rating is stored with nitrogen overlay and used within one month.

The skilled artisan will appreciate that the structures embrace the natural isotope form, as well as other stable isotopes. For example, but not limited to, hydrogen may be deuterated.

Antimicrobial preservatives are reported to induce instability in formulations through various hypotheses of interactions between peptides and preservatives. It is possible that a combination of mechanisms contributes to the interaction between peptide and preservative simultaneously.

Static light scattering and solution NMR techniques were used to study the interaction between tirzepatide and preservatives.

### Assays:

### Light Scattering of Tirzepatide Samples

Tirzepatide formulations were prepared for light scattering measurements, with compositions detailed in Tables 1.a and 1.b. Light scattering data were collected using an ALV-CGS3 goniometer-based light scattering instrument (ALV-GmbH, Langen, Germany), which is a self-contained system with 22 mW 633 nm HeNe laser. Samples were filtered into disposable glass tubes using appropriate filters. Corresponding drug product placebos were used as scattering blanks. A single 15- or 30-second-long acquisition at 90-degree scattering angle was collected per sample, using the dynamic light scattering (DLS) mode. The resulting DLS auto-correlation functions were analyzed using intensity-weighted regularized size distributions, and shown to be monomodal before proceeding. The time-averaged scattering intensity values of the samples, placebos and toluene were used to calculate the excess Rayleigh ratios.

Apparent weight-average molecular weight (WAMW) values were calculated using formula (1):

$$WAMW = {[ ( K \cdot c ) / R ]}^{- 1} \qquad ( 1 )$$

where K is an optical constant, c is the peptide mass concentration, and R is the excess Rayleigh ratio. A refractive index increment (dn/dc) of 0.185 mL/g was assumed.

TABLE 1a

| Tirzepatide | Phosphate | Tonicity | | |
|---|---|---|---|---|
| (mg/mL) | Buffer | agent | Preservative ^{a} | pH ^{b} |
| 30 | 5 mM | — | — | 7.0 |
| | (1.34 | | m-Cresol, 3.15 mg/mL | |
| | mg/mL) | | Phenol, 5.5 mg/mL | |
| | | | Benzyl alcohol, | |
| | | | 9 mg/mL + | |
| | | | phenol, 2 mg/mL | |
| | | NaCl, 140 | — | |
| | | mM (8.18 | m-Cresol, 3.15 mg/mL | |
| | | mg/mL) | Phenol, 5.5 mg/mL | |
| | | | Benzyl alcohol, | |
| | | | 9 mg/mL + | |
| | | | phenol, 2 mg/mL | |
| | | — | | |
| | | NaCl, 200 | — | |
| | | mM (11.7 | | |
| | | mg/mL) | | |
| | | NaCl, 10 | | |
| | | mM (0.585 | Benzyl alcohol, | |
| | | mg/mL) | 9 mg/mL + | |
| | | NaCl, 50 | phenol, 2 mg/mL | |
| | | mM (2.925 | | |
| | | mg/mL) | | |

TABLE 1b

| Tirzepatide | Phosphate | NaCl | | |
|---|---|---|---|---|
| (mg/mL) | buffer | (mM) | Preservative | pH |
| 0 | 5 mM | 50 | None | 7.0 |
| 40 | (1.34 | (2.93 | | 7.0 |
| | mg/mL) | mg/mL) | | |
| 0 | 5 mM | 50 | Benzyl alcohol, 9 | 7.0 |
| 40 | (1.34 | (2.93 | mg/mL + phenol, 2 | 7.0 |
| | mg/mL) | mg/mL) | mg/mL | |
| 0 | 5 mM | 0 | None | 7.0 |
| 40 | (1.34 | | | 7.0 |
| | mg/mL) | | | |
| 0 | 5 mM | 0 | Phenol, 5.5 mg/mL | 7.0 |
| 40 | (1.34 | | | 7.0 |
| | mg/mL) | | | |
| 0 | 5 mM | 20 | None | 7.0 |
| 40 | (1.34 | (1.17 | | 7.0 |
| | mg/mL) | mg/mL) | | |
| 0 | 5 mM | 20 | Phenol, 5.5 mg/mL | 7.0 |
| 40 | (1.34 | (1.17 | | 7.0 |
| | mg/mL) | mg/mL) | | |

### Thioflavin T (ThT) Fluorescence Assay of Tirzepatide Samples

ThT fluorescence was collected using a SpectraMax Gemini EM Microplate Reader (Molecular Devices, San Jose, California). For each sample or placebo (Table 2), 40 mL of solution was added to each of three wells in a black, clear bottom 96-well plate, followed by adding 10 mL of 20 mM ThT stock solution. The plate was sealed, and a 24-hour kinetic fluorescence measurement with 450 nm excitation and 480 nm emission wavelengths was performed. The temperature was set to 37° C., and measurements were taken every 10 minutes with 3 seconds of shaking prior to each measurement. A positive control sample (Oxyntomodulin, 2 mg/mL, pH=6.5) was included in the measurement.

TABLE 2
Compositions of tirzepatide formulations for ThT fluorescence

| Tirzepatide | Phosphate | Tonicity | | |
|---|---|---|---|---|
| (mg/mL) | Buffer | agent | Preservative | pH |
| 15 | 5 mM | Glycerin | m-Cresol, 3.15 mg/mL | 7.0 |
| | (1.34 | (17 mg/mL) | Phenol, 5.5 mg/mL | |
| | mg/mL) | | Benzyl alcohol, 9 mg/mL + | |
| | | | phenol, 2 mg/mL | |
| | | NaCl, 140 | m-Cresol, 3.15 mg/mL | |
| | | mM (8.18 | Phenol, 5.5 mg/mL | |
| | | mg/mL) | Benzyl alcohol, 9 mg/mL + | |
| | | | phenol, 2 mg/mL | |
| 0 | 5 mM | Glycerin | Phenol, 5.5 mg/mL | 7.0 |
| 5 | (1.34 | (17 mg/mL) | | |
| 30 | mg/mL) | | | |
| 0 | | | Benzyl alcohol, 9 mg/mL + | |
| 5 | | | phenol, 2 mg/mL | |
| 30 | | | | |
| 0 | | | Phenol, 5.5 mg/mL | |
| 5 | | NaCl, 140 | | |
| 30 | | mM (8.18 | | |
| 0 | | mg/mL) | Benzyl alcohol, 9 mg/mL + | |
| 5 | | | phenol, 2 mg/mL | |
| 30 | | | | |

### Effect of Preservative and NaCl on the Self-Association of Tirzepatide

The higher order structure of tirzepatide was characterized using far-UV circular dichroism (CD) spectroscopy and Fourier-transform infrared spectroscopy (FTIR). In the Mounjaro™ matrix (5 mM phosphate buffer, 140 mM NaCl, pH=7.0), tirzepatide has a predominantly α-helical structure. Using light scattering, the self-association of tirzepatide between the dose and concentration ranges of 2.5 mg/0.5 mL (5 mg/mL) and 15 mg/0.5 mL (30 mg/mL) is shown to exist as a monomer-trimer-hexamer equilibrium.

To study the effect of preservatives, m-cresol, phenol or benzyl alcohol at selected concentrations were added to a standard Mounjaro™ composition. These concentrations of the preservatives were selected such that the formulations would meet the passing criteria of antimicrobial efficacy test. Results from the light scattering experiments are summarized in Table 3. The Mounjaro™ composition (“Sample 5”) shows a WAMW of 23.6 kDa. This value is consistent with the previously described monomer-trimer-hexamer equilibrium.

In the presence of both 140 mM NaCl and preservatives, the WAMW of tirzepatide was determined to be 70 kDa or larger, consistent with 14-mer to 16-mer oligomers (Table 3, “Samples 6 to 8”). Correspondingly, the solution appeared opalescent, as opposed to the “clear” appearance of Mounjaro™ solutions.

TABLE 3
Effect of preservatives on the self-association of tirzepatide

| | Tonicity | | WAMW | |
|---|---|---|---|---|
| Sample #: ^{a, b} | agent | Preservative(s) | (kDa) | Association ^{c} |
| −1 | None | None | 5.2 | 1 |
| −2 | | m-Cresol, 3.15 mg/mL | 7.0 | 1 |
| −3 | | Phenol, 5.5 mg/mL | 7.0 | 1 |
| −4 | | Benzyl alcohol, 9 | 6.9 | 1 |
| | | mg/mL + phenol, 2 | | |
| | | mg/mL | | |
| −5 | NaCl, 140 | None | 23.6 | 5 |
| −6 | mM | m-Cresol, 3.15 mg/mL | 69.5 | 14 |
| −7 | (8.18 mg/mL) | Phenol, 5.5 mg/mL | 73.6 | 15 |
| −8 | | Benzyl alcohol, 9 | 78.0 | 16 |
| | | mg/mL + phenol, 2 | | |
| | | mg/mL | | |

### Fibrillation Propensity of Tirzepatide in the Presence of Preservatives

Fibrils are large macromolecular self-assemblies of proteins or peptides with specific characteristics. Most notable is the conversion of the individual peptide backbone into a β-sheet-enriched conformation. As a result, undesired physical, chemical, and therapeutic risks can be raised. Experimentally, fibril formation may be visually observed as increased turbidity, precipitation, or gelation, and may also be studied using a plethora of techniques ranging from size exclusion chromatography, analytical ultracentrifugation, light scattering, and microscopic imaging. These methods can be complemented by spectroscopy, such that on a molecular level, the conformational change towards β-sheet and the formation of larger assemblies can be confirmed.

In the current study, the risk of tirzepatide fibrillation in the presence of preservatives was evaluated using fluorescence spectroscopy, with thioflavin T (ThT) as the binding dye. ThT is a potent fluorescent marker of fibrils. Once selectively bound to fibril deposits, the fluorescence signal exhibits a dramatic increase, indicating the presence or increase of fibrils. This method has been applied to a variety of peptides to study their fibrillation propensity. Tirzepatide was formulated in matrices containing different tirzepatide concentrations, tonicity agents and preservatives (Table 2). A positive control known to fibrillate (Oxyntomodulin, 2 mg/mL, pH=6.5) was included in the measurement. An increase in the ThT fluorescence signal can clearly be seen with the positive control. On the other hand, none of the tirzepatide samples showed any statistically significant increase in the signal, indicating very low risk of fibrillation.

Size Exclusion Chromatography (SEC) Shelf-Life and in-Use Stability Study

This procedure is an isocratic size exclusion HPLC method with UV detection at 214 nm and is designed to determine the relative amounts of high molecular weight species. The SEC column is a 125 Å SEC column, 3.5 μm particle size, 7.8 mm×300 mm or equivalent. The mobile phase is 50/50/0.05% acetonitrile/water/TFA and the flow rate is 0.5 mL/min. The column temperature is 25° C. High molecular weight species are reported as peak area percent to the total area. The procedure is stability indicating as measured by its ability to resolve known impurities from tirzepatide. This study compares alternate compositions with the compositions embodiments herein.

### RP-HPLC Shelf Life and in Use Stability Study

This procedure is a gradient reversed-phase HPLC method employing a C18 reversed-phase column with dimensions of 2.6 μm, 4.6×250 mm, or equivalent, with UV detection at 214 nm. Mobile Phase A is 0.1% trifluoroacetic acid in water and mobile phase B is 0.1% trifluoroacetic acid in acetonitrile (ACN) with the gradient profile presented in Table 4.

TABLE 4
RP-HPLC gradient profile.

| Time | Flow | 0.1% TFA in | 0.1% TFA in |
|---|---|---|---|
| (minutes) | (mL/min) | H₂O | ACN |
| 0 | 1.0 or 1.2 | 75.0 | 25.0 |
| 0.1 | 1.2 | 75.0 | 25.0 |
| 1.0 | 1.2 | 75.0 | 25.0 |
| 11.0 | 1.2 | 57.5 | 42.5 |
| 51.0 | 1.2 | 47.5 | 52.5 |
| 52.0 | 1.2 | 10.0 | 90.0 |
| 54.0 | 1.2 | 10.0 | 90.0 |
| 54.1 | 1.2 | 75.0 | 25.0 |
| 59.8 | 1.2 | 75.0 | 25.0 |
| 59.9 | 1.0 or 1.2 | 75.0 | 25.0 |
| 60.0 | 1.0 or 1.2 | 75.0 | 25.0 |

The column temperature is controlled at 60° C. and the method is designed to determine the assay, identity, and purity of tirzepatide in the drug product. Identity is determined by matching the retention time of the main peak with that of the main peak of an external reference standard. Assay is determined by the comparison of the main peak area with the corresponding peak in the external reference standard. Impurities and related substances are reported as peak area percent to the total peak area. The procedure is stability indicating as judged by its ability to resolve known impurities from tirzepatide. A composition comprising 30 mM to 50 mM NaCl, 8 mg/mL glycerin; 5 mM phosphate buffer, and phenol as a preservative can provide acceptable in use stability.

### Stability Studies

The primary stability studies include six batches of multiple-dose tirzepatide injection drug product in the prefilled pen (PFP) configuration. Drug product from each of these batches was filled into 3-mL clear glass cartridges sealed on one side with an elastomeric plunger and on the other side with a disc seal consisting of a bilayer elastomeric disc and an aluminum shell.

Samples from each batch were placed on stability at the 5° C. (2° C.-8° C.) long-term storage condition for at least 24 months, and at the accelerated condition of 25° C./60% relative humidity (RH) for 6 months. Additionally, samples were stored at the stress stability condition of 30° C./65% RH. The stability protocol for the drug product is outlined in Table 5.

TABLE 5
Primary Stability Protocol for the Drug Product

| Analytical | Storage | Time Point (Months)^{a} | | | | | | | |
|---|---|---|---|---|---|---|---|---|---|
| Property | Conditions | 0 | 1 | 3 | 6 | 9 | 12 | 18 | 24 |
| Purity | 5° C. | X | — | X | X | X | X | X | X |
| Related Substances/ | 25° C./60% RH | | X | X | X | — | — | — | — |
| Impurities (RP-LC): | 30° C./65% RH | | X | X | X | — | — | — | — |
| Total | | | | | | | | | |
| L-isoAsp15 | | | | | | | | | |
| L-isoAsp9 | | | | | | | | | |
| D-Ser32/C-Term | | | | | | | | | |
| Deamidation | | | | | | | | | |
| Gln19 Deamidation | | | | | | | | | |
| Gln24 Deamidation | | | | | | | | | |
| High Molecular | 5° C. | X | — | X | X | X | X | X | X |
| Weight Species | 25° C./60% RH | | X | X | X | — | — | — | — |
| | 30° C./65% RH | | X | X | X | — | — | — | — |
| Benzyl Alcohol | 5° C. | X | — | X | X | X | X | X | X |
| | 25° C./60% RH | | X | X | X | — | — | — | — |
| Phenol | 5° C. | X | — | X | X | X | — | X | X |
| | 25° C./60% RH | | X | X | X | — | — | — | — |
| Description | 5° C. | X | — | X | X | X | X | X | X |
| | 25° C./60% RH | | X | X | X | — | — | — | — |
| Color^{b} | 5° C. | X | — | X | X | X | X | X | X |
| | 25° C./60% RH | | X | X | X | — | — | — | — |
| Clarity^{b} | 5° C. | X | — | X | X | X | X | X | X |
| | 25° C./60% RH | | X | X | X | — | — | — | — |
| Sterility | 5° C. | X | — | X | — | — | X | — | X |
| Container Closure | 5° C. | X | — | X | — | — | X | — | X |
| Integrity^{c} | | | | | | | | | |
| pH | 5° C. | X | — | X | X | X | X | X | X |
| | 25° C./60% RH | | X | X | X | — | — | — | — |
| | 30° C./65% RH | | X | X | X | — | — | — | — |
| Part. Matter | 5° C. | X | — | X | X | X | X | X | X |
| ≥10 μm | 25° C./60% RH | | X | X | X | — | — | — | — |
| Part. Matter | 30° C./65% RH | | X | X | X | — | — | — | — |
| ≥25 μm | | | | | | | | | |
| Dose Accuracy | 5° C. | X | — | X | X | X | X | X | X |
| | 25° C./60% RH | | X | X | X | — | — | — | — |
| Injection | 5° C. | X | — | X | X | X | X | X | X |
| (Glide) Force | 25° C./60% RH | | X | X | X | — | — | — | — |

In addition to the long-term (5° C.), accelerated (25° C./60% RH), and stress (30° C./65% RH) conditions studied as summarized in Table 5, an in-use stability study was conducted to demonstrate acceptable stability over the period of time during which the multiple-dose drug product can be utilized by the patient out of refrigeration. For this study, two primary stability batches were selected following a concentration bracketing approach whereby one was selected at the low end (2.5 mg/0.6 mL) and one at the high end (15 mg/0.6 mL) of the intended drug product concentration ranges. The study was conducted on prefilled pen batches with concentrations of 2.5 mg/0.6 mL and 15 mg/0.6 mL after storage at 2° C. to 8° C. for approximately 2-3 months¹. For the in-use study, both batches were stored at the in-use condition of 30° C. and tested over a period of 30 days, as shown in Table 6 below. At 4 timepoints within the in-use study (0, 14, 22, and 30 days), samples were removed from 30° C. storage and a needle was affixed to the prefilled pen. After priming, drug product solution was expelled from the prefilled pen, after which the needle was removed, and samples were either tested or returned to 30° C. for expulsion and testing at later timepoints. In this manner, samples reaching the end of the in-use study had been exposed to conditions (e.g., 30° C.) and manipulation (e.g., affixing a needle, expelling drug product solution, removing the needle) simulating the intended use by the patient. The protocol for multi-dose product stability is shown in Table 6.

TABLE 6
In-Use Primary Stability Protocol for the Drug Product

| | Storage | In-Use Timepoint at 30° C. (Days) | | | |
|---|---|---|---|---|---|
| Analytical Property | Condition | 0^{a} | 14 | 22 | 30 |
| Purity | 30° C. | X | X | X | X |
| Related Substances/ | | | | | |
| Impurities: | | | | | |
| Total | | | | | |
| L-isoAsp15 | | | | | |
| L-isoAsp9 | | | | | |
| D-Ser32/C-Terminal | | | | | |
| Deamidation | | | | | |
| Gln19 Deamidation | | | | | |
| Gln24 Deamidation | | | | | |
| High Molecular | 30° C. | X | X | X | X |
| Weight Species | | | | | |
| Benzyl Alcohol | 30° C. | X | X | X | X |
| Phenol | 30° C. | X | X | X | X |
| Description | 30° C. | X | X | X | X |
| Color | 30° C. | X | X | X | X |
| Clarity | 30° C. | X | X | X | X |
| pH | 30° C. | X | X | X | X |
| Part. Matter ≥10 μm | 30° C. | X | X | X | X |
| Part. Matter ≥25 μm | | | | | |
| Dose Accuracy | 30° C. | X | X | X | X |
| Injection (Glide) Force | 30° C. | X | X | X | X |
| Antimicrobial | 30° C. | — | — | X | X |
| Effectiveness | | | | | |
| Testing (AET) | | | | | |

^{a}T0 is the date the samples are removed from the long-term storage condition of 5° C. and tested, i.e., 30 days prior to end of the in-use study.

These tests include those attributes of the drug product that are susceptible to change during storage and may influence quality, safety, and/or efficacy. Color, clarity, and pH were tested to confirm that, like the single-dose product, meaningful changes are not observed on stability and that they are not shelf-life limiting attributes. Antimicrobial effectiveness testing (AET) was conducted on samples exposed to the in-use conditions. The AET testing combined with the preservative content testing demonstrates the microbial safety of the drug product during the in-use period.

### RP-HPLC Purity

Purity was evaluated at the long-term (5° C.), accelerated (25° C./60% RH), and stress stability (30° C./65% RH) storage conditions. At the accelerated condition, the batches show a slight decrease in purity. Based on the available data, all batches met the end of shelf-life acceptance criteria at the long-term and accelerated storage conditions.

### Total Impurities

Total impurities was evaluated at the long-term (5° C.), accelerated (25° C./60% RH), and stress stability (30° C./65% RH) storage conditions. At the accelerated condition, the batches show a slight increase in total impurities. Based on the available data, all batches met the end of shelf-life acceptance criteria at the long-term and accelerated storage conditions.

### High Molecular Weight Species

High molecular weight species (HMWS) were evaluated at the long-term (5° C.), accelerated (25° C./60% RH), and stress stability (30° C./65% RH) storage conditions. At the accelerated condition, the batches show a slight increase in HMWS. Based on the available data, all batches met the end of shelf-life acceptance criteria for HMWS at the long term and accelerated storage conditions.

### Benzyl Alcohol

Benzyl alcohol content was evaluated at the long-term (5° C.) and accelerated (25° C./60% RH) storage conditions. At the accelerated condition, the data show no distinct trend on stability for benzyl alcohol. Based on the available data, all batches met the end of shelf-life acceptance criteria for benzyl alcohol at the long term and accelerated storage conditions.

### Phenol

Phenol content was evaluated at the long-term (5° C.) and accelerated (25° C./60% RH) storage conditions. At the accelerated condition, the data show no distinct trend on stability for phenol. Based on the available data, all batches met the end of shelf-life acceptance criteria for phenol at the long term and accelerated storage conditions.

### Color

Color was evaluated at the long-term (5° C.) and accelerated (25° C./60% RH) storage conditions using a series of reference solutions. Based on the available data, all batches met the acceptance criteria for color at both the long-term and accelerated conditions. Color is not a shelf-life limiting parameter for this composition.

### Clarity

Clarity was evaluated at the long-term (5° C.) and accelerated (25° C./60% RH) storage conditions using the instrumental (Ratio Turbidimetry) methods. No trending or significant variability outside of method variability was observed.

### Sterility

All primary stability batches met sterility requirements. Container closure integrity (CCI) may be used in lieu of sterility testing. All available CCI results from the primary stability studies were “Pass”.

pH

pH was evaluated at the long-term (5° C.), accelerated (25° C./60% RH), and stress stability. Based on the available data, all batches met the end of shelf-life acceptance criteria for pH at both the long-term and accelerated conditions. No trending in results was observed, demonstrating that pH is well controlled throughout storage.

### Particulate Matter

Particulate matter was evaluated at the long-term (5° C.), accelerated (25° C./60% RH), and stress stability (30° C./65% RH) storage conditions. All batches in the study met the requirements at both the long-term and accelerated conditions.

### Injection (Glide) Force

Injection (Glide) force was evaluated at the long-term (5° C.) and accelerated (25° C./60% RH) storage conditions using compression testing. No trending or significant variability outside of method variability was observed at either condition. All batches in the study met the acceptance criteria for injection force at both the long-term and accelerated conditions. Injection force is not expected to be a shelf-life limiting parameter.

### Dose Accuracy

Dose accuracy was evaluated at the long-term (5° C.) and accelerated (25° C./60% RH) storage conditions using gravimetric volume by weight method. No trending or significant variability outside of method variability was observed at either condition. All batches in the study met the acceptance criteria for dose accuracy at both the long-term and accelerated conditions. Dose accuracy is not expected to be a shelf-life limiting parameter.

### Antimicrobial Effectiveness Test (AET)

Characteristics Studied, an antimicrobial effectiveness test (AET) was conducted on samples from on in-use samples from the 22- and 30-day timepoints. the in-use study to verify the effectiveness of the antimicrobial preservatives. Testing was performed according to USP on in-use samples from the 22- and 30-day timepoints. These results support that the multi-dose product meets compendial AET criteria after storage at the in-use condition for the full in-use period, as well as at the end of the in-use period after storage at the accelerated stability condition for 6 months. Results support a shelf-life for the multiple-dose tirzepatide injection drug product of 24 months when stored at the long-term storage condition of about 5° C. (2° C.-8° C.) with an in-use period of 30 days up to about 30° C. In-use stability results are summarized by Table 7.

TABLE 7
In-Use Stability Results (30° C.)

| | Concentration | In-Use Timepoint at 30° C. (Days) | | | |
|---|---|---|---|---|---|
| Test | (mg/0.6 mL) | T = 0 | T = 14 | T = 22 | T = 30 |
| Purity (%) | 2.5 | 97.0 | 96.3 | 96.2 | 96.0 |
| | 15 | 97.0 | 96.2 | 96.0 | 95.4 |
| Total Impurities (%) | 2.5 | 3.0 | 3.7 | 3.8 | 4.0 |
| | 15 | 3.0 | 3.8 | 4.0 | 4.6 |
| L-isoAsp15 (%) | 2.5 | <0.1% | 0.2 | 0.3 | 0.4 |
| | 15 | 0.1 | 0.3 | 0.3 | 0.4 |
| L-isoAsp9 (%) | 2.5 | 0.4 | 0.7 | 0.8 | 0.9 |
| | 15 | 0.6 | 0.8 | 0.9 | 1.1 |
| D-Ser32/C-Term Deamidation (%) | 2.5 | 0.7 | 0.8 | 0.8 | 0.8 |
| | 15 | 0.6 | 0.7 | 0.7 | 0.8 |
| Gln19 Deamidation (%) | 2.5 | 0.5 | 0.5 | 0.5 | 0.5 |
| | 15 | 0.5 | 0.5 | 0.6 | 0.6 |
| Gln24 Deamidation (%) | 2.5 | 0.1 | 0.1 | 0.1 | 0.1 |
| | 15 | 0.1 | 0.1 | 0.1 | 0.1 |
| HMWS (%) | 2.5 | 0.2 | 0.2 | 0.2 | 0.3 |
| | 15 | 0.2 | 0.2 | 0.3 | 0.3 |
| Benzyl Alcohol (% Label Claim) | 2.5 | 102.4 | 101.7 | 108.2 | 108.1 |
| | 15 | 101.4 | 101.0 | 108.2 | 109.0 |
| Phenol (% Label Claim) | 2.5 | 102.7 | 102.5 | 100.1 | 99.5 |
| | 15 | 102.2 | 101.8 | 100.5 | 100.8 |
| Description | 2.5 | Pass | Pass | Pass | Pass |
| | 15 | Pass | Pass | Pass | Pass |
| pH | 2.5 | 7.1 | 7.1 | 7.1 | 7.1 |
| | 15 | 7.2 | 7.2 | 7.2 | 7.1 |
| HIAC ≥10 μm (particles/mL) | 2.5 | 7 | 5 | 75 | 30 |
| | 15 | 44 | 46 | 32 | 19 |
| HIAC ≥25 μm (particles/mL) | 2.5 | 0 | 0 | 0 | 0 |
| | 15 | 0 | 0 | 0 | 1 |
| HIAC ≥2 μm (particles/mL) | 2.5 | 326 | 539 | 422 | 226 |
| | 15 | 818 | 464 | 375 | 220 |
| HIAC ≥5 μm (particles/mL) | 2.5 | 50 | 81 | 204 | 103 |
| | 15 | 249 | 175 | 137 | 74 |
| MFI ≥2 μm (particles/mL) | 2.5 | 10808 | 1883 | 1625 | 4394 |
| | 15 | 504 | 2950 | 427 | 744 |
| MFI ≥5 μm (particles/mL) | 2.5 | 919 | 287 | 162 | 239 |
| | 15 | 123 | 645 | 44 | 128 |
| Color (Ph. Eur. 2.2.2) | 2.5 | <BY7 | <BY7 | <BY7 | <BY7 |
| | 15 | <BY7 | <BY7 | <BY7 | <BY7 |
| Clarity (Ph. Eur. 2.2.1) | 2.5 | 0.71 | 0.64 | 0.65 | 0.69 |
| | 15 | 2.00 | 2.08 | 2.20 | 2.04 |
| Dose Accuracy - | 2.5 | Pass | Pass | Pass | Pass |
| (Mean)^{a} - mL | | (0.60) | (0.60) | (0.59) | (0.60) |
| | 15 | Pass | Pass | Pass | Pass |
| | | (0.60) | (0.60) | (0.60) | (0.60) |
| Injection (Glide) Force - | 2.5 | Pass | Pass | Pass | Pass |
| (Mean)^{a} - N | | (16.9) | (16.9) | (16.9) | (16.7) |
| | 15 | Pass | Pass | Pass | Pass |
| | | (17.0) | (17.2) | (16.9) | (16.7) |

^{a}Testing is completed on a representative sample per an acceptance sampling plan defined by internal quality standards. The mean values are reported for reference only.

Results of storage stability for 0 and 3 months at storage temperature of 2 to 8 (are shown in Table 8.

TABLE 8
Long Term Condition Stability

| | | Time (Months) | |
|---|---|---|---|
| Test | Units | Initial | 3 |
| Assay (RP-LC-UV) | % | 101.0 | 100.3 |
| Purity (RP-LC-UV) | % | 97.5 | 97.5 |
| Related Substances/Impurities | % | 2.5 | 2.5 |
| (RP-LC-UV): | | | |
| Total | | | |
| L-isoAsp15 | % | <0.1 | 0.1 |
| L-isoAsp9 | % | 0.4 | 0.5 |
| D-Ser32/C-term Deamidation | % | 0.5 | 0.6 |
| Gln19 Deamidation | % | 0.4 | 0.3 |
| Gln24 Deamidation | % | 0.2 | 0.2 |
| High Molecular Weight | % | 0.3 | 0.2 |
| Species (SEC) | | | |
| Description | NA | Pass* | Pass* |
| Color (Ph. Eur. 2.2.2) | NA | Colorless | Colorless |
| Clarity (Ph. Eur. 2.2.1 | NTU | 0 | 1 |
| Instrumental) | | | |
| Sterility | NA | Pass | Pass |
| Container Closure Integrity*** | N/A | Pass | Pass |
| pH | N/A | 7.1 | 7.1 |
| Particulate | Part./ | 142 | 184 |
| Matter ≥ 10 μm (HIAC) | Container | | |
| Particulate | Part./ | 0 | 3 |
| Matter ≥ 25 μm (HIAC) | Container | | |
| Benzyl Alcohol | | 102.1 | 101.7 |
| Phenol | | 103.0 | 102.3 |

*Pass = Clear, colorless solution, free of visible particles.
***CCI testing is performed on cartridge batch D589770.

### NMR Studies and Self-Association

Tirzepatide formulations were prepared for light scattering measurements and NMR, with compositions detailed in Tables 9, 10, and 11.

TABLE 9
Compositions of tirzepatide formulations for static light scattering

| | Tirzepatide | | Tonicity | | |
|---|---|---|---|---|---|
| Solution | (mg/mL) | Buffer | agent | Preservative ^{a} | pH |
| 1 | 0 to approximately | Phosphate, 5 | NaCl, 140 | None | 7.0 |
| | 30 | mM | mM | | |
| 2 | 0 to approximately | Phosphate, 5 | NaCl, 140 | Phenol, liquefied, | 7.0 |
| | 30 | mM | mM | distilled, 5 mg/mL | |
| 3 | 0 to approximately | Phosphate, 5 | NaCl, 0 | None | 7.0 |
| | 30 | mM | mM | | |
| 4 | 0 to approximately | Phosphate, 5 | NaCl, 0 | Phenol, liquefied, | 7.0 |
| | 30 | mM | mM | distilled, 5 mg/mL | |
| 5 | 0 to approximately | Phosphate, 5 | NaCl, 0 | Glycerin, 24.4 mg/mL | 7.0 |
| | 30 | mM | mM | | |
| 6 | 0 to approximately | Phosphate, 5 | NaCl, 30 | None | 7.0 |
| | 30 | mM | mM | | |
| 7 | 0 to approximately | Phosphate, 5 | NaCl, 85 | None | 7.0 |
| | 30 | mM | mM | | |
| 8 | 0 to approximately | Phosphate, 5 | NaCl, 200 | None | 7.0 |
| | 30 | mM | mM | | |
| 9 | 0 to approximately | Phosphate, 5 | NaCl, 0 | Phenol, liquefied, | 7.0 |
| | 30 | mM | mM | distilled, 8 mg/mL | |
| 10 | 0 to approximately | Phosphate, 5 | NaCl, 30 | Phenol, liquefied, | 7.0 |
| | 30 | mM | mM | distilled, 5 mg/mL | |
| 11 | 0 to approximately | Phosphate, 5 | NaCl, 30 | Phenol, liquefied, | 7.0 |
| | 30 | mM | mM | distilled, 8 mg/mL | |
| 12 | 0 to approximately | Phosphate, 5 | NaCl, 0 | Benzyl alcohol, 9 | 7.0 |
| | 30 | mM | mM | mg/mL | |
| 13 | 0 to approximately | Phosphate, 5 | NaCl, 30 | Benzyl alcohol, 9 | 7.0 |
| | 30 | mM | mM | mg/mL | |
| 14 | 0 to approximately | Phosphate, 5 | NaCl, 0 | Benzyl alcohol, 15 | 7.0 |
| | 30 | mM | mM | mg/mL | |
| 15 | 0 to approximately | Phosphate, 5 | NaCl, 30 | Benzyl alcohol, 15 | 7.0 |
| | 30 | mM | mM | mg/mL | |
| 16 | 0 to approximately | Phosphate, 5 | NaCl, 0 | Benzyl alcohol, 9 | 7.0 |
| | 30 | mM | mM | mg/mL + | |
| | | | | Phenol, liquefied, | |
| | | | | distilled, 2 mg/mL | |
| 17 | 0 to approximately | Phosphate, 5 | NaCl, 30 | Benzyl alcohol, 9 | 7.0 |
| | 30 | mM | mM | mg/mL + | |
| | | | | Phenol, liquefied, | |
| | | | | distilled, 2 mg/mL | |

TABLE 10
Compositions of tirzepatide formulations for NMR

| Tirzepatide | Buffer and | | |
|---|---|---|---|
| (mg/mL) | tonicity | Preservative | pH |
| 0 | Phosphate (5 mM) | Benzyl alcohol, 6 mg/mL | 7.0 |
| 0.5 | and NaCl (30 | Benzyl alcohol, 0.3 mg/mL | 7.0 |
| 0.5 | mM) | Benzyl alcohol, 1.8 mg/mL | 7.0 |
| 1 | | Benzyl alcohol, 0.6 mg/mL | 7.0 |
| 1 | | Benzyl alcohol, 3.6 mg/mL | 7.0 |
| 5 | | Benzyl alcohol, 0 mg/mL | 7.0 |
| 5 | | Benzyl alcohol, 3 mg/mL | 7.0 |
| 5 | | Benzyl alcohol, 6 mg/mL | 7.0 |
| 0 | Phosphate (5 mM) | Phenol, liquefied, distilled, 10 mg/mL | 7.0 |
| 0.5 | and NaCl (30 mM) | Phenol, liquefied, distilled, 0.025 mg/mL | 7.0 |
| 0.5 | | Phenol, liquefied, distilled, 1.5 mg/mL | 7.0 |
| 1 | | Phenol, liquefied, distilled, 0.05 mg/mL (0.056 | 7.0 |
| | | mg/mL) | |
| 1 | | Phenol, liquefied, distilled, 3 mg/mL | 7.0 |
| 5 | | Phenol, liquefied, distilled, 0.25 mg/mL (0.28 | 7.0 |
| | | mg/mL) | |
| 5 | | Phenol, liquefied, distilled, 10 mg/mL | 7.0 |
| 20 | Phosphate (5 mM) | None | 7.0 |
| 0 | and NaCl (30 mM) | None | 7.0 |

TABLE 11
Compositions of tirzepatide formulations for NMR

| Tirzepatide | Buffer and | | |
|---|---|---|---|
| (mg/mL) | tonicity | Preservative | pH |
| 5 | Phosphate (5 mM) and | Phenol, liquefied, distilled, | 7.0 |
| 15 | NaCl (30 mM) | 2 mg/mL | 7.0 |
| 25 | | | 7.0 |
| 25 | | None | 7.0 |

### EXAMPLES

### Example Formulation(s) for Preserved Tirzepatide

Preserved formulation examples are set forth in Table 12. In addition to the 50 mM NaCl, appropriate amount of glycerin is added as the second tonicity agent. Formulations are studied verify the antimicrobial efficacy of the formulations, as well as desired physical and chemical stability of the drug product.

TABLE 12
Compositions for the preserved tirzepatide drug product

| | Tirzepatide | | Tonicity | | |
|---|---|---|---|---|---|
| Example | (mg/mL) | Buffer | agent | Preservative | pH |
| 1 | 5 to 30 | Phosphate, | NaCl, 50 mM | Benzyl alcohol, | 7.0 |
| | | 5 mM (1.34 | (2.93 mg/mL) + | 9 mg/mL + | |
| | | mg/mL) | Glycerin, | Phenol, 2 | |
| | | | 8 mg/mL | mg/mL | |
| 2 | | | NaCl, 50 mM | Phenol, 5.5 | 7.0 |
| | | | (2.93 mg/mL) + | mg/mL | |
| | | | Glycerin, | | |
| | | | 12 mg/mL | | |

The concentration of NaCl plays a critical role in dictating the association state of tirzepatide, i.e., higher WAMW with increasing NaCl concentration. Self-association of the peptide becomes amplified when preservatives are added. At 50 mM NaCl in formulation of Examples 1 and 2, the oligomerization state of tirzepatide is considered equivalent as that in the Mounjaro™ matrix. These results support that the risk of tirzepatide forming fibrils in a 50 mM NaCl preserved formulation is low.

A synergistic effect was discovered at 50 mM NaCl, in formulations of Examples 1 and 2. At 50 mM NaCl, Example 1 and 2, the oligomerization state of tirzepatide is considered equivalent as that in the Mounjaro™ matrix at the 30 mg/mL tirzepatide.

### Examples 3 and 4

The formulation of Examples 3 and 4 are prepared substantially as described herein, as set forth by Table 13.

TABLE 13

| | Tirzepatide | | Tonicity | | |
|---|---|---|---|---|---|
| Formulation | (mg/mL) ^{a} | Buffer | agent ^{b} | Preservative | pH |
| Example 3 | 4.17 to 25 | Phosphate, | NaCl, | Benzyl alcohol, | 7.0 |
| | | 5 mM | 30 mM | 9 mg/mL + | |
| | | (or 1.34 | (or 1.75 | phenol, 2 mg/mL | |
| Example 4 | | mg/mL) | mg/mL) + | Phenol, with a | 7.0 |
| | | | Glycerin, | concentration >5.5 | |
| | | | 8 mg/mL | mg/ml ^{c} | |

^{a}The concentration of tirzepatide in the preserved formulation is adjusted based on the 0.6-mL injection volume. The dose range of 2.5 mg to 15 mg remains unchanged. As shown below, the concentration depends on the injection volume.

An example of tirzepatide concentrations using formulations herein, appear in Table 14.

TABLE 14

| | Mounjaro ™ | | Preserved tirzepatide formulation | |
|---|---|---|---|---|
| Dose | | Concentration | | Concentration |
| (mg) | Presentation | (mg/mL) | Presentation | (mg/mL) |
| 2.5 | 2.5 mg/0.5 mL | 5 | 2.5 mg/0.6 mL | 4.17 |
| 5 | 5 mg/0.5 mL | 10 | 5 mg/0.6 mL | 8.33 |
| 7.5 | 7.5 mg/0.5 mL | 15 | 7.5 mg/0.6 mL | 12.50 |
| 10 | 10 mg/0.5 mL | 20 | 10 mg/0.6 mL | 16.67 |
| 12.5 | 12.5 mg/0.5 mL | 25 | 12.5 mg/0.6 mL | 20.83 |
| 15 | 15 mg/0.5 mL | 30 | 15 mg/0.6 mL | 25.00 |

### EMBODIMENTS

Embodiment 1. A pharmaceutical composition comprising SEQ ID NO:2, or a pharmaceutically acceptable salt thereof; NaCl; glycerin; phenol; and phosphate buffer; wherein the NaCl concentration is less than or equal to about 3 mg/mL.

2. A pharmaceutical composition of embodiment 1 wherein the phosphate buffer concentration is about 5 mM.

3. A pharmaceutical composition of any one of embodiment 1 or 2 wherein the phosphate buffer concentration is about 1.34 mg/mL.

4. A pharmaceutical composition of any one of embodiments 1 to 3 wherein the NaCl concentration is from about 30 mM to about 50 mM.

5. A pharmaceutical composition of any one of embodiments 1 to 4 wherein the NaCl concentration is about 30 mM.

6. A pharmaceutical composition of any one of embodiments 1 to 5 wherein the NaCl concentration is about 1.75 mg/mL.

7. A pharmaceutical composition of any one of embodiments 1 to 6 wherein the glycerin concentration is from about 8 mg/mL to 12 mg/mL.

8. A pharmaceutical composition of any one of embodiments 1 to 7 wherein the glycerin concentration is about 8 mg/mL.

9. A pharmaceutical composition of any one of embodiments 1 to 8 wherein the phenol concentration is from about 1.5 mg/mL to about 6 mg/mL.

10. A pharmaceutical composition of any one of embodiments 1 to 9 wherein the phenol concentration is from about 2 mg/mL to about 5.5 mg/mL.

11. A pharmaceutical composition of any one of embodiments 1 to 10 wherein the phenol concentration is about 5.5 mg/mL.

12. A pharmaceutical composition of any one of embodiments 1 to 11 wherein the composition further comprises benzyl alcohol.

13. A pharmaceutical composition of any one of embodiments 1 to 12 wherein the preservative comprises phenol and benzyl alcohol.

14. A pharmaceutical composition of any one of embodiments 1 to 12 wherein the preservative comprises about 9 mg/mL benzyl alcohol.

15. A pharmaceutical composition of embodiment 14 wherein the preservative comprises about 2 mg/mL phenol and about 9 mg/mL benzyl alcohol.

16. A pharmaceutical composition of any one of embodiments 1 to 15 wherein the preservative comprises low peroxide benzyl alcohol.

17. A pharmaceutical composition of any one of embodiments 1 to 16 wherein the preservative comprises benzyl alcohol with less than about 4 parts per million peroxide.

18. A pharmaceutical composition of any one of embodiments 1 to 17 wherein the pH of the composition is from about 6.5 to about 7.5.

19. A pharmaceutical composition of any one of embodiments 1 to 18 wherein the pH of the composition is from about 6.7 to about 7.3.

20. A pharmaceutical composition of any one of embodiments 1 to 19 wherein the composition is about pH 7.

21. A pharmaceutical composition of embodiment 1 wherein:
phosphate buffer concentration is about 5 mM; NaCl is about 2.93 mg/ml; Phenol is about 2 mg/mL; Glycerin is about 8 mg/mL; and benzyl alcohol is about 9 mg/mL.

22. A pharmaceutical composition of embodiment 1 wherein Phosphate buffer concentration is about 5 mM; NaCl concentration is about 30 mM Glycerin; and Phenol.

23. A pharmaceutical composition of embodiment 22 wherein the glycerin is about 8 mg/mL.

24. A pharmaceutical composition of any one of embodiments 22 to 23 wherein the phenol is about 2 mg/mL to about 5.5 mg/mL

25. A pharmaceutical composition of any one of embodiments 22 to 24 wherein the phenol is about 5.5 mg/mL.

26. A pharmaceutical composition of any one of embodiments 22 to 24 wherein the phenol is about 2 mg/mL.

27. A pharmaceutical composition of any one of embodiments 1 to 26 wherein the composition is presented in a multiuse injection device.

28. A pharmaceutical composition of any one of embodiments 1 to 26 wherein the composition is administered using a multiuse injection device.

Embodiment 29. A pharmaceutical composition comprising SEQ ID NO:3, or a pharmaceutically acceptable salt thereof; NaCl; glycerin; phenol; and phosphate buffer; wherein the NaCl concentration is less than or equal to about 3 mg/mL.

30. A pharmaceutical composition of embodiment 29 wherein the phosphate buffer concentration is about 5 mM.

31. A pharmaceutical composition of any one of embodiment 29 or 30 wherein the phosphate buffer concentration is about 1.34 mg/mL.

32. A pharmaceutical composition of any one of embodiments 29 to 31 wherein the NaCl concentration is from about 30 mM to about 50 mM.

33. A pharmaceutical composition of any one of embodiments 29 to 32 wherein the NaCl concentration is about 30 mM.

34. A pharmaceutical composition of any one of embodiments 29 to 33 wherein the NaCl concentration is about 1.75 mg/mL.

35. A pharmaceutical composition of any one of embodiments 29 to 34 wherein the glycerin concentration is from about 8 mg/mL to 12 mg/mL.

36. A pharmaceutical composition of any one of embodiments 29 to 35 wherein the glycerin concentration is about 8 mg/mL.

37. A pharmaceutical composition of any one of embodiments 29 to 36 wherein the phenol concentration is from about 1.5 mg/mL to about 6 mg/mL.

38. A pharmaceutical composition of any one of embodiments 29 to 37 wherein the phenol concentration is from about 2 mg/mL to about 5.5 mg/mL.

39. A pharmaceutical composition of any one of embodiments 29 to 38 wherein the phenol concentration is about 5.5 mg/mL.

40. A pharmaceutical composition of any one of embodiments 29 to 39 wherein the composition further comprises benzyl alcohol.

41. A pharmaceutical composition of any one of embodiments 29 to 40 wherein the preservative comprises phenol and benzyl alcohol.

42. A pharmaceutical composition of any one of embodiments 29 to 41 wherein the preservative comprises about 9 mg/mL benzyl alcohol.

43. A pharmaceutical composition of embodiment 42 wherein the preservative comprises about 2 mg/mL phenol and about 9 mg/mL benzyl alcohol.

44. A pharmaceutical composition of any one of embodiments 29 to 43 wherein the preservative comprises low peroxide benzyl alcohol.

45. A pharmaceutical composition of any one of embodiments 29 to 44 wherein the preservative comprises benzyl alcohol with less than about 4 parts per million peroxide.

46. A pharmaceutical composition of any one of embodiments 29 to 45 wherein the pH of the composition is from about 6.5 to about 7.5.

47. A pharmaceutical composition of any one of embodiments 29 to 46 wherein the pH of the composition is from about 6.7 to about 7.3.

48. A pharmaceutical composition of any one of embodiments 29 to 47 wherein the composition is about pH 7.

49. A pharmaceutical composition of embodiment 29 wherein:
phosphate buffer concentration is about 5 mM; NaCl is about 2.93 mg/ml; Phenol is about 2 mg/mL; Glycerin is about 8 mg/mL; and benzyl alcohol is about 9 mg/mL.

50. A pharmaceutical composition of embodiment 29 wherein Phosphate buffer concentration is about 5 mM; NaCl concentration is about 30 mM Glycerin; and Phenol.

51. A pharmaceutical composition of embodiment 50 wherein the glycerin is about 8 mg/mL.

52. A pharmaceutical composition of any one of embodiments 50 to 51 wherein the phenol is about 2 mg/mL to about 5.5 mg/mL.

53. A pharmaceutical composition of any one of embodiments 50 to 52 wherein the phenol is about 5.5 mg/mL.

54. A pharmaceutical composition of any one of embodiments 50 to 52 wherein the phenol is about 2 mg/mL.

55. A pharmaceutical composition of any one of embodiments 29 to 54 wherein the composition is presented in a multiuse injection device.

56. A pharmaceutical composition of any one of embodiments 29 to 54 wherein the composition is administered using a multiuse injection device.

Embodiment 57. A pharmaceutical composition comprising SEQ ID NO:4, or a pharmaceutically acceptable salt thereof; NaCl; glycerin; phenol; and phosphate buffer; wherein the NaCl concentration is less than or equal to about 3 mg/mL.

58. A pharmaceutical composition of embodiment 57 wherein the phosphate buffer concentration is about 5 mM.

59. A pharmaceutical composition of any one of embodiment 57 or 58 wherein the phosphate buffer concentration is about 1.34 mg/mL.

60. A pharmaceutical composition of any one of embodiments 57 to 59 wherein the NaCl concentration is from about 30 mM to about 50 mM.

61. A pharmaceutical composition of any one of embodiments 57 to 60 wherein the NaCl concentration is about 30 mM.

62. A pharmaceutical composition of any one of embodiments 57 to 61 wherein the NaCl concentration is about 1.75 mg/mL.

63. A pharmaceutical composition of any one of embodiments 57 to 62 wherein the glycerin concentration is from about 8 mg/mL to 12 mg/mL.

64. A pharmaceutical composition of any one of embodiments 57 to 63 wherein the glycerin concentration is about 8 mg/mL.

65. A pharmaceutical composition of any one of embodiments 57 to 64 wherein the phenol concentration is from about 1.5 mg/mL to about 6 mg/mL.

66. A pharmaceutical composition of any one of embodiments 57 to 65 wherein the phenol concentration is from about 2 mg/mL to about 5.5 mg/mL.

67. A pharmaceutical composition of any one of embodiments 57 to 66 wherein the phenol concentration is about 5.5 mg/mL.

68. A pharmaceutical composition of any one of embodiments 57 to 67 wherein the composition further comprises benzyl alcohol.

69. A pharmaceutical composition of any one of embodiments 57 to 68 wherein the preservative comprises phenol and benzyl alcohol.

70. A pharmaceutical composition of any one of embodiments 57 to 69 wherein the preservative comprises about 9 mg/mL benzyl alcohol.

71. A pharmaceutical composition of embodiment 70 wherein the preservative comprises about 2 mg/mL phenol and about 9 mg/mL benzyl alcohol.

72. A pharmaceutical composition of any one of embodiments 57 to 71 wherein the preservative comprises low peroxide benzyl alcohol.

73. A pharmaceutical composition of any one of embodiments 57 to 72 wherein the preservative comprises benzyl alcohol with less than about 4 parts per million peroxide.

74. A pharmaceutical composition of any one of embodiments 57 to 73 wherein the pH of the composition is from about 6.5 to about 7.5.

75. A pharmaceutical composition of any one of embodiments 57 to 74 wherein the pH of the composition is from about 6.7 to about 7.3.

76. A pharmaceutical composition of any one of embodiments 57 to 75 wherein the composition is about pH 7.

77. A pharmaceutical composition of embodiment 57 wherein:
phosphate buffer concentration is about 5 mM; NaCl is about 2.93 mg/ml; Phenol is about 2 mg/mL; Glycerin is about 8 mg/mL; and benzyl alcohol is about 9 mg/mL.

78. A pharmaceutical composition of embodiment 57 wherein Phosphate buffer concentration is about 5 mM; NaCl concentration is about 30 mM Glycerin; and Phenol.

79. A pharmaceutical composition of embodiment 78 wherein the glycerin is about 8 mg/mL.

80. A pharmaceutical composition of any one of embodiments 78 to 79 wherein the phenol is about 2 mg/mL to about 5.5 mg/mL.

81. A pharmaceutical composition of any one of embodiments 78 to 80 wherein the phenol is about 5.5 mg/mL.

82. A pharmaceutical composition of any one of embodiments 78 to 80 wherein the phenol is about 2 mg/mL.

83. A pharmaceutical composition of any one of embodiments 57 to 82 wherein the composition is presented in a multiuse injection device.

84. A pharmaceutical composition of any one of embodiments 57 to 82 wherein the composition is administered using a multiuse injection device.

### Sequences

Tirzepatide
SEQ ID NO: 1
YX₁EGTFTSDYSIX₂LDKIAQKAFVQWLIAGGPSSGAPPPS

wherein X₁ is Aib; X₂ is Aib; K at position 20 is chemically modified through conjugation to the epsilon-amino group of the K side-chain with
(2-[2-(2-Amino-ethoxy)-ethoxy]-acetyl)₂-(γGlu)₁-CO—(CH₂)₁₈—CO₂H;
and the C-terminal amino acid is amidated as a C-terminal primary amide.

SEQ ID NO: 2
YX₁EGTFTSDYSIX₂LDKIAQKAFVQWLIAGGPSSGAPPPS

wherein X₁ is Aib; X₂ is Aib; K at position 20 is chemically modified through conjugation to the epsilon-amino group of the K side-chain with a C16-C20 fatty acid or a derivative thereof; and the C-terminal amino acid is optionally amidated as a C-terminal primary amide.

SEQ ID NO: 3
YX₁EGTFTSDYSIX₂LDKIAQKAFVQWLIAGGPSSGAPPPS

(SEQ ID NO: 3) wherein X₁ is Aib; X₂ is Aib; K at position 20 is chemically modified through conjugation to the epsilon-amino group of the K side-chain with a fatty acid selected from the group consisting of

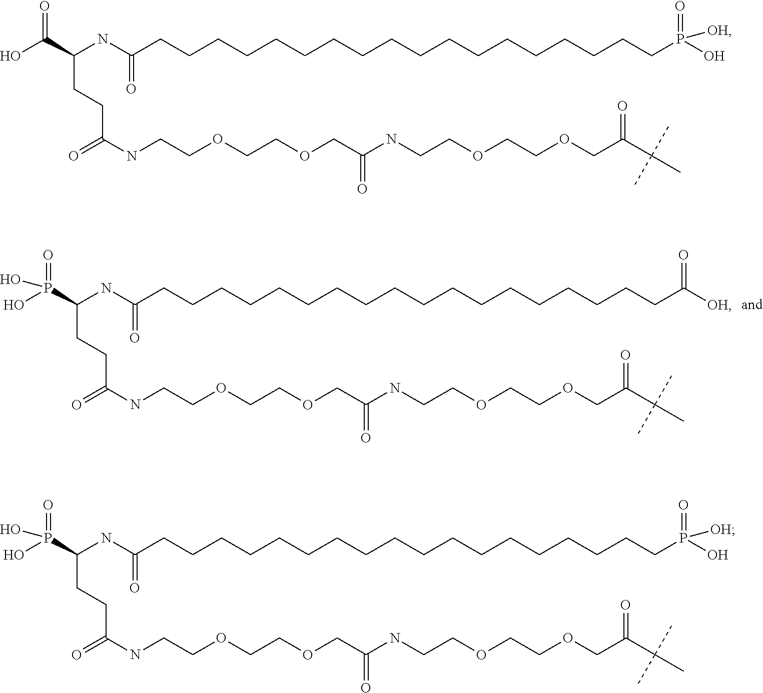

and the C-terminal amino acid is optionally amidated as a C-terminal primary amide: or a pharmaceutically acceptable salt thereof.

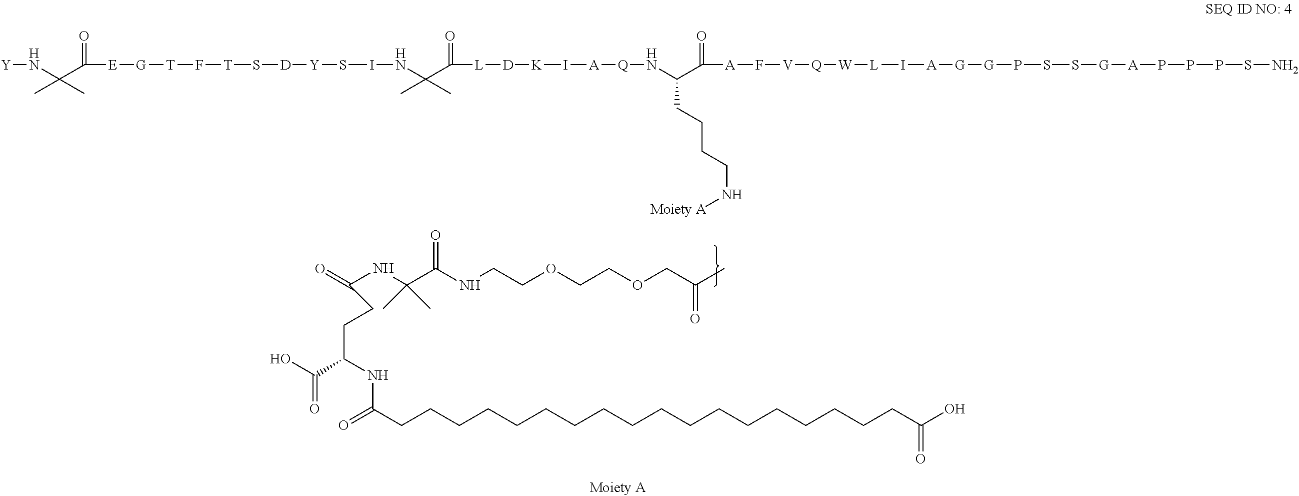

We claim:

1. A pharmaceutical composition comprising tirzepatide, or a pharmaceutically acceptable salt thereof; NaCl; glycerin; phenol; and phosphate buffer; wherein the NaCl concentration is less than or equal to about 3 mg/mL.

2. A pharmaceutical composition as claimed by claim 1, wherein the tirzepatide, or a pharmaceutically acceptable salt thereof, concentration is from about 2 to about 50 mg/mL.

3. A pharmaceutical composition as claimed by any one of claim 1 or 2, wherein the tirzepatide, or a pharmaceutically acceptable salt thereof, concentration is selected from the group consisting of about 5, about 10, about 15, about 20, about 25, about 30, about 40, and about 50 mg/mL.

4. A pharmaceutical composition as claimed claim 1 wherein the tirzepatide, or pharmaceutically acceptable salt thereof, concentration is selected from the group consisting of about 10, about 20, and about 30 mg/mL.

5. A pharmaceutical composition as claimed by claim 2, wherein the tirzepatide or a pharmaceutically acceptable salt thereof, concentration is from about 4.2 to about 41.8 mg/mL.

6. A pharmaceutical composition as claimed by claim 5 wherein the tirzepatide, or pharmaceutically acceptable salt thereof, concentration is selected from the group consisting of about 4.17, about 8.33, about 12.5, about 16.67, about 20.83, about 25, about 33.33, and about 41.8 mg/mL.

7. A pharmaceutical composition as claimed by any one of claim 5 or 6 wherein the tirzepatide, or pharmaceutically acceptable salt thereof, concentration is selected from the group consisting of about 4.17, about 8.33, about 12.50, about 16.67, about 20.83, and about 25 mg/mL.

8. A pharmaceutical composition as claimed by any one of claims 1 to 7 in a unit dose presentation, wherein the unit dose of tirzepatide, or pharmaceutically acceptable salt thereof, is selected from the group consisting of about 5, about 7.5, about 10, about 12.5, about 15, about 20, and about 25 mg.

9. A pharmaceutical composition as claimed by any one of claims 1 to 7 in a unit dose presentation, wherein the unit dose of tirzepatide, or a pharmaceutically acceptable salt thereof, is selected from the group consisting of 5, 7.5, 10, 12.5, and 15 mg.

10. A pharmaceutical composition as claimed by any one of claims 1 to 9 wherein the dose volume administered is about 0.5 mL.

11. A pharmaceutical composition as claimed by any one of claims 1 to 9 wherein the dose volume administered is about 0.6 mL.

12. A pharmaceutical composition as claimed by any one of claims 1 to 8 wherein the phosphate buffer concentration is about 5 mM.

13. A pharmaceutical composition as claimed by any one of claims 1 to 8 wherein the phosphate buffer concentration is about 1.34 mg/mL.

14. A pharmaceutical composition as claimed by any one of claims 1 to 8 wherein the NaCl concentration is from about 30 mM to about 50 mM.

15. A pharmaceutical composition as claimed by claim 14 wherein the NaCl concentration is about 30 mM.

16. A pharmaceutical composition as claimed by claim 1 wherein the NaCl concentration is about 1.75 mg/mL.

17. A pharmaceutical composition as claimed by any one of claims 1 to 8 or claims 12 to 16—wherein the glycerin concentration is from about 8 mg/mL to about 12 mg/mL.

18. A pharmaceutical composition as claimed by claim 17 wherein the glycerin concentration is about 8 mg/mL.

19. A pharmaceutical composition as claimed by any one of claims 1 to 8 or claims 12 to 16 wherein the phenol concentration is from about 1.5 mg/mL to about 6 mg/mL.

20. A pharmaceutical composition as claimed by claim 19 by wherein the phenol concentration is from about 2 mg/mL to about 5.5 mg/mL.

21. A pharmaceutical composition as claimed by claim 20 wherein the phenol concentration is about 5.5 mg/mL.

22. A pharmaceutical composition as claimed by any one of claims 1 to 8 or claims 12 to 16 wherein the composition further comprises benzyl alcohol

23. A pharmaceutical composition as claimed by claim 22 wherein the composition further comprises about 9 mg/mL benzyl alcohol.

24. A pharmaceutical composition as claimed by claim 22 wherein the composition further comprises low peroxide grade benzyl alcohol.

25. A pharmaceutical composition as claimed by claim 22 wherein the composition further comprises benzyl alcohol with less than about 4 parts per million peroxide.

26. A pharmaceutical composition as claimed claim 1 wherein the composition pH is from about 6.5 to about 7.5.

27. A pharmaceutical composition as claimed by claim 26 wherein the composition pH is from about 6.7 to about 7.3.

28. A pharmaceutical composition as claimed by claim 26 wherein the composition pH is about 7.

29. A pharmaceutical composition as claimed by claim 1 wherein
tirzepatide, or a pharmaceutically acceptable salt thereof, concentration is from about 5 to about 30 mg/mL; the phosphate buffer concentration is about 5 mM; NaCl is about 2.93 mg/mL; Phenol is about 5.5 mg/mL; and Glycerin is about 12 mg/mL.

30. A pharmaceutical composition as claimed by claim 1 wherein:
tirzepatide, or a pharmaceutically acceptable salt thereof, concentration is from about 5 to about 30 mg/mL; phosphate buffer concentration is about 5 mM; NaCl is about 2.93 mg/mL; Phenol is about 2 mg/mL; Glycerin is about 8 mg/mL; and further comprising about 9 mg/mL benzyl alcohol.

31. A pharmaceutical composition as claimed by claim 1 wherein tirzepatide, or a pharmaceutically acceptable salt thereof, concentration is from about 4.17 mg/mL to about 25 mg/mL; Phosphate buffer concentration is about 5 mM; NaCl concentration is about 30 mM; Glycerin; and Phenol.

32. A pharmaceutical composition as claimed by claim 31 wherein the glycerin concentration is about 8 mg/mL.

33. A pharmaceutical composition as claimed by any one of claims 31 to 32 wherein the phenol concentration is about 2 mg/mL to about 5.5 mg/mL

34. A pharmaceutical composition as claimed by claim 33 wherein the phenol concentration is about 5.5 mg/mL.

35. A pharmaceutical composition as claimed by claim 33 wherein the phenol concentration is about 2 mg/mL.

36. A pharmaceutical composition as claimed by claim 31 wherein the composition further comprises benzyl alcohol.

37. A pharmaceutical composition as claimed claim 36 wherein the composition further comprises about 9 mg/mL benzyl alcohol.

38. A pharmaceutical composition as claimed by any one of claims 1 to 37 wherein the composition is stable for at least 3 months at a temperature between 2 to 8 degrees Celsius.

39. A pharmaceutical composition as claimed by any one of claims 1 to 38 wherein the composition is stable for at least 6 months at a temperature between 2 to 8 degrees Celsius.

40. A pharmaceutical composition as claimed by any one of claims 1 to 39 wherein the composition is stable for at least 24 months at a temperature between 2 to 8 degrees Celsius.

41. A pharmaceutical composition as claimed by any one of claims 1 to 40 wherein the composition is stable during in use for at least 30 days at 30 degrees Celsius.

42. A pharmaceutical composition as claimed by any one of claims 1 to 41 wherein the composition is stable, as measured using high molecular weight species.

43. A pharmaceutical composition as claimed by any one of claims 1 to 42 wherein the composition is presented in a multiuse injection device.

44. A pharmaceutical composition as claimed by any one of claims 1 to 42 wherein the composition is administered using a multiuse injection device.

45. A method for treating type 2 diabetes in a human in need thereof, comprising administering an effective amount of a composition as claimed by any one of claims 1 to 8.

46. A method for treating type 2 diabetes as claimed by claim 45 wherein the dose is administered once weekly.

47. A method for improving chronic weight management in a human in need thereof, comprising administering an effective amount of a composition as claimed by any one of claims 1 to 8 or claims 12 to 16.

48. A method as claimed by claim 47 wherein the human in need thereof is obese.

49. A pharmaceutical composition as claimed by any one of claims 1 to 8 or claims 12 to 16 for use in the treatment of type 2 diabetes.

50. A pharmaceutical composition as claimed by any one of claims 1 to 8 or claims 12 to 16 for use to improve chronic weight management.

51. A pharmaceutical composition as claimed by any one of claims 1 to 8 or claims 12 to 16 for use in treating obesity.
